(19) United States
(12) Patent Application Publication
(10) Pub. No.: US 2014/0046044 A1
(43) Pub. Date: Feb. 13, 2014

(54) NOVEL GLYCOSYL PHOSPHITES

(71) Applicants: Ignacio Pérez Figueroa, Miami, FL (US); Ferenc Horváth, Pillisszentkereszt (HU); Gyula Dekany, Sinnamon Park (AU); Christian Risinger, Rottweil (DE); Markus Hederos, Svedala (SE)

(72) Inventors: Ignacio Pérez Figueroa, Miami, FL (US); Ferenc Horváth, Pillisszentkereszt (HU); Gyula Dekany, Sinnamon Park (AU); Christian Risinger, Rottweil (DE); Markus Hederos, Svedala (SE)

(73) Assignee: GLYCOM A/S, Kongens Lyngby (DK)

(21) Appl. No.: 14/000,574

(22) PCT Filed: Feb. 21, 2012
(86) PCT No.: PCT/DK2012/050059

§ 371 Date: Aug. 20, 2013

(30) Foreign Application Priority Data
Feb. 21, 2011 (DK) PA 2011 70092
May 13, 2011 (EP) 11166063.5
May 13, 2011 (EP) 11166135.1
May 13, 2011 (EP) 11166137.7

(51) Int. Cl. C07H 11/04; C07H 3/06

(52) U.S. Cl. CPC C07H 11/04; C07H 3/06
USPC 536/17.1; 536/123.1

(57) ABSTRACT

The present invention relates to providing compounds of general formula 1

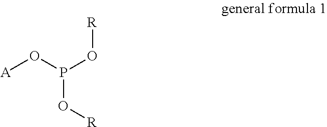

wherein A is glycosyl residue of a mono-, di- or oligosaccharide in protected form and R is selected from optionally substituted aryl or optionally substituted heteroaryl, methods for their preparation and their use in glycosidation reactions.

### FIELD OF THE INVENTION

The present invention provides novel glycosyl phosphites, methods for their preparation and their use in glycosidation reactions.

### BACKGROUND OF THE INVENTION

Glycosyl phosphites have been known for two decades and their advantageous features in glycosylation reactions have been recognized early. Generally, glycosyl phosphites can be synthesized in a straightforward manner, they are relatively stable, easy to handle and require common activators/promoters for glycosylations avoiding thus to resort to the use of specific, precious or toxic heavy-metal salts. It can be utilized in the construction of both 1,2-trans-β- and 1,2-cis-α-glycosidic linkages. Appearance of sialyl-phosphites in sialic acid chemistry has made progression in α-selective sialylation which has always been a problematic area due to several factors: the presence of the carboxylic moiety at the anomeric position that creates unfavoured steric effect for stereoselective α-sialylation, 2,3-elimination may be triggered due to the electron-withdrawing effect of the carboxylic portion and the lack of participating group at C-3.

Chemistry of glycosyl phosphites including sialyl phosphites has been reviewed extensively [e.g. S, Nakamura et al. in: Handbook of Chemical Glycosylation (ed. A. V. Demchenko), Wiley, 2008, pp. 223-259; Z. Zhang et al. in: Carbohydrates in Chemistry and Biology (eds. B. Ernst, G. W. Hart, P. Sinaÿ), Wiley, 2000. Vol. 1, pp. 117-134, D. K. Ress et al. Curr. Org. Synth. 1, 31 (2004)].

Some dialkyl-, dibenzyl-, pinacolyl- and o-xylylene-phosphitylated glycosyl derivatives have been synthesized and employed in glycosylation reactions. Although it is difficult to isolate glycosyl phosphites in anomerically pure form, diethyl and dibenzyl phosphite of sialic acid could be obtained in pure β-form. Nevertheless, it is not only the anomeric purity of the donor that directs the stereochemical outcome of the glycosylation reactions, but several other factors such as the solvent effect, promoter design, donor-acceptor match and neighbouring group participation.

Due to a number of parameters mentioned above that affects the stereoselectivity of glycosylation reactions, no universal methodology for interglycosidic bond formation could be elaborated. Thus there is always a need to develop further solutions that solve particular problems and overcome hurdles arisen.

### SUMMARY OF THE INVENTION

The first aspect of the present invention relates to compounds of general formula 1

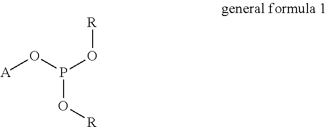

wherein A is glycosyl residue of a mono-, di- or oligosaccharide in protected form and R is selected from optionally substituted aryl or optionally substituted heteroaryl.

The second aspect of the present invention provides a method for producing a compound of general formula 1 according to the first aspect, characterized in that a compound of formula A-OH, wherein A means a protected glycosyl residue of a mono-, di- or oligosaccharide is
a) reacted with a compound (RO)₂PY wherein R is selected from optionally substituted aryl and optionally substituted heteroaryl, and Y is selected from halogen and dialkylamino, or
b) reacted with a compound PX₃ wherein X is halogen, followed by reaction with an alcohol ROH wherein R is defined as above.

The third aspect of the present invention relates to a process for the synthesis of an oligosaccharide, characterized in that the said synthesis comprises at least the step of: coupling a compound of general formula 1 according to the first aspect with an acceptor of the formula B—OH, wherein B—OH means a protected mono-, di- or oligosaccharide.

### DETAILED DESCRIPTION OF THE INVENTION

Throughout the present description, the term “alkyl”, either alone or when attached to another atom or group, means a linear or branched hydrocarbon group with 1-20 carbon atoms, preferably with 1-6 carbon atoms, like methyl, ethyl, n-propyl, i-propyl, n-butyl, i-butyl, s-butyl, t-butyl, etc. The term “cycloalkylidene” means a bivalent cyclic hydrocarbon ring having 3-8 carbon atoms, such as cyclopropylidene, cyclopentylidene, cyclohexylidene, cycloheptylidene, etc.

In the present application the term “aryl” refers to homoaromatic groups like phenyl or naphthyl. Preferably, aryl means phenyl.

In the present application the term “heteroaryl” refers to aromatic groups having one or two rings, which ring(s) contain(s) 1, 2, or 3 heteroatoms selected from the group of N, O and S, such as pyrrol, imidazole, pyrazole, 1,2,3-triazole, 1,2,4-triazole, furan, thiophene, oxazole, isoxazole, thiazole, thiadiazole, pyridine, pyrazine, pyridazine, pyrimidine, triazine, benzimidazole, benzoxazole, benzthiazole, indole, quinoline, isoquinoline, purine, pteridine and the like.

In the present description, the term “acyl” represent an R*—C(═O)—, wherein R* may be H, alkyl or aryl, like formyl, acetyl, propionyl, butyryl, pivaloyl, benzoyl, etc. The alkyl and aryl residues both may be substituted.

Herein, the term “group removable by hydrogenolysis” means a protecting group whose C—O bond to the oxygen can be cleaved by hydrogen in the presence of a catalytic amount of palladium, Raney nickel or any other conventional hydrogenolysis catalyst to regenerate the OH group. Such protecting groups are described in Wuts and Greene: Protective Groups in Organic Synthesis, John Wiley & Sons, 2007, and include benzyl, diphenylmethyl(benzhydryl), 1-naphthylmethyl, 2-naphthylmethyl and triphenylmethyl(trityl) groups, each of which can be optionally substituted by one or more of the following groups: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). A preferred protecting group is benzyl optionally substituted with one or more of the following groups: phenyl, alkyl and halogen, particularly unsubstituted benzyl, 4-chlorobenzyl, 3-phenylbenzyl and 4-methylbenzyl groups.

For the purpose of this specification with claims, the term “optionally substituted” means that the group in question may either carry a substituent or may be unsubstituted.

For the purpose of this specification with claims, the term “substituted” means that the group in question is substituted with a group which modifies the general chemical characteristics of the chain or ring. The substituents can be used to modify characteristics of the molecule as a whole, such as stability, solubility, and ability to form crystals. The person skilled in the art will be aware of other suitable substituents of a similar size and charge characteristics, which could be used as alternatives in a given situation.

More generally in connection with the terms “alkyl”, “cycloalkylidene”, “aryl”, “heteroaryl” and “acyl” the term “optionally substituted” is intended to mean that the group in question may be substituted one or several times, preferably 1-5 times, more preferably 1-3 times with group(s) selected from the group consisting of alkyl (only for aryl, heteroaryl and aromatic acyl), hydroxy, alkoxy (i.e. alkyl-oxy), carboxy, oxo (forming a keto or aldehyde functionality), alkoxycarbonyl, alkylcarbonyl, formyl, aryl, aryloxycarbonyl, aryloxy, arylamino, arylcarbonyl, amino, mono- and dialkylamino, carbamoyl, mono- and dialkyl-aminocarbonyl, alkylcarbonylamino, cyano, alkanoyloxy, nitro, alkylthio and halogen (F, Cl, Br, I).

The present invention relates to the novel compounds of general formula 1

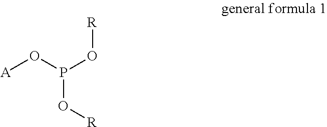

wherein A is glycosyl residue of a mono-, di- or oligosaccharide in protected form and R is selected from optionally substituted aryl or optionally substituted heteroaryl.

When R denotes optionally substituted heteroaryl, it is attached to the oxygen atom by a carbon atom of the aromatic ring.

The term “glycosyl residue of a mono-, di- or oligosaccharide in protected form” intends to mean any derivatized or non-derivatized mono-, di- or oligosaccharide glycosyl residue which is attached to the —O—P(OR)₂ phosphityl group by the C-1 (aldoses) or C-2 (ketoses) anomeric carbon atom thus forming glycosyl phosphite type compounds. If the glycosyl residue differs from monosaccharide, it may represent a linear or branched structure consisting of monosaccharide units that are linked to each other by interglycosidic linkages. The monosaccharide or monosaccharides units can be selected from any 5-9 carbon atom containing sugars consisting of aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglycosamine, N-acetylmannosamine, N-acetylgalactosamine, etc.), uronic acids, ketoaldonic acids (e.g. sialic acid) and like. The functional groups of the glycosyl residue are protected and/or derivatized. The protective groups can be the commonly used ones in organic/carbohydrate chemistry; they are well known to the skilled man and are discussed e.g. in P. G. M. Wuts and T. W. Greene: Protective Groups in Organic Synthesis, John Wiley & Sons (2007); S. Hanessian: Preparative Carbohydrate Chemistry Marcel Dekker (1997); Chemical Synthesis of Glycosides and Glycomimetics in: Carbohydrates in Chemistry and Biology (Eds.: B. Ernst, G. W. Hart, P. Sinaÿ) Part I, Vol. 1, Wiley (2000).

According to a preferred embodiment of compounds of general formula 1, A means a sialyl moiety in protected form or a fucosyl moiety in protected form, such as a sialyl moiety in protected form, in protected form and R is optionally substituted aryl.

The term “sialyl moiety in protected form” refers to glycosyl residue of any naturally occurring or modified neuraminic or sialic acid derivatives and analogues thereof in protected form. In a sialyl moiety the C-2 (anomeric) carbon atom is attached to the phosphityl residue. Preferred neuraminic acids are N-acetyl- (Neu5Ac), N-glycolyl- (Neu5Gc) and deamino-neuraminic acid (3-deoxy-D-glycero-D-galacto-nonulosonic acid, KDN). Also included are Neu5Ac, Neu5Gc and KDN derivatives that are derivatized with linkers, reactive functional groups, detectable labels or targeting moieties. The derivatization may affect C-3 with the introduction of bulky thio groups, C-4 with the introduction of piperidino, piperazino or morpholino moieties and C-5 with the introduction of azido group and formation of 5-N,4-O cyclic carbamate. The protective groups on the sialyl moiety are to mask hydroxyls (as ethers and/or esters and/or acetals), —NHAc or —NH₂ (as amides or carbamates) and the carboxylic portion (as esters or thioesters) and commonly used in organic/carbohydrate chemistry. Thus C-5 nitrogen can be protected e.g. as N,N-diacetyl, N-trifluoroacetyl, N-trichloroacetyl, N-phthalyl, N-Troc, N-Fmoc and the like.

The term “fucosyl moiety in protected form” refers to a fucopyranosyl moiety attached to the phosphityl residue via the anomeric carbon atom, and in which the hydroxyl groups are protected as ethers and/or esters and/or acetals or by other means commonly used in organic/carbohydrate chemistry.

A more preferred embodiment encompasses compounds of general formulae 2A and 2B, such as compounds of general formula 2A,

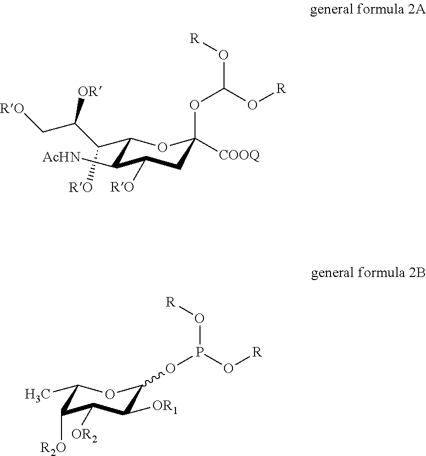

wherein R is optionally substituted phenyl, R′ is optionally substituted acyl, Q is optionally substituted alkyl, R₁ is a group removable by hydrogenolysis or optionally substituted acyl, and R₂ is a group removable by hydrogenolysis, optionally substituted acyl or two R₂ groups together form a moiety

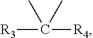

wherein R₃ and R₄, independently, are alkyl or phenyl, or wherein groups R₃ and R₄ together with the carbon atom to which they are attached form cycloalkylidene.

An especially preferred embodiment relates to compounds of general formulae 3A and 3B, such as compounds of general formula 3A,

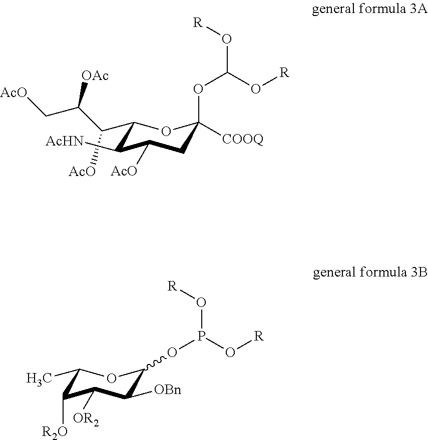

wherein Q is selected from C₁₋₆ alkyl and benzyl, preferably methyl, R is phenyl optionally substituted with alkyl, alkoxy and/or halogen, preferably methyl, methoxy and/or bromo, and R₂ is benzyl, acetyl or benzoyl optionally substituted with chloro

Unlike to other glycosyl phosphites specified in the art, compounds of general formula 1, preferably of general formulae 2A and 2B, even more preferably of general formulae 3A and 3B, such as of general formula 3A, of the present application can be considered as crystalline materials. They are stable, can be stored for longer period of time without significant decomposition, can be easily activated in glycosylation reactions and show excellent α-selectivity. Accordingly, compounds of general formulae 3A and 3B, such as of general formula 3A, according to the present application have obviously advantageous applicability in sialylation or fucosylation reactions, such as in sialylation reactions.

Another aspect of the present application is a method for producing compounds of general formula 1, characterized in that the compound of formula A-OH, wherein A means glycosyl residue of a mono-, di- or oligosaccharide in protected form is reacted with
a) a compound of (RO)₂PY in which R is selected from optionally substituted aryl and optionally substituted heteroaryl and Y is selected from halogen and dialkylamino, or
b) PX₃ wherein X is halogen, followed by reaction with an alcohol ROH wherein R is defined as above.

In a compound of formula A-OH A is glycosyl residue of a mono-, di- or oligosaccharide in protected form as defined above, thus compound of A-OH represents any derivatized or non-derivatized protected mono-, di- or oligosaccharide in protected form with free glycosidic OH.

The reactions are typically carried out in aprotic solvent or mixture aprotic solvents, such as halogenated solvents like dichloromethane or chloroform, DMF, dioxane, toluene, acetonitrile, etc. When conducting the synthesis according to option a) tertiary amines like triethyl amine or Hünig's base are employed to scavenge the liberating acid HY. As to option b) in the first step N-containing aromatic bases like pyridine, imidazole, tetrazole are the preferred base of choice then in the subsequent condensation step tertiary amines like triethyl amine or Hünig's base are used to neutralize acids. The two types of base can be added to the reaction mixture in the same time or consecutively.

In a preferred method option b) is taken wherein the first reaction is carried out at 0-25° C., preferably at 5-10° C., then the second reaction is conducted at 20-40° C., preferably at room temperature. As to reactants and reagents, the preferred anomerically free sugar is a suitably protected sialic acid derivative, more preferably N-acetyl neuraminic acid tetraacetate methyl ester, PX₃ is PCl₃ and the aromatic alcohol ROH is phenol optionally substituted with alkyl, alkoxy and/or halogen, preferably methyl, methoxy and/or bromo.

In another preferred method option b) is taken wherein the first reaction is carried out at 0-25° C., preferably at 5-10° C., then the second reaction is conducted at 20-40° C., preferably at room temperature. As to reactants and reagents, the preferred anomerically free sugar is a suitably protected fucose derivative, preferably 2-O-benzyl-3,4-di-O-(optionally substituted acyl)-L-fucose, PX₃ is PCl₃ and the aromatic alcohol ROH is phenol optionally substituted with alkyl, alkoxy and/or halogen, preferably methyl, methoxy and/or bromo.

Compounds of general formulae 1, 2A, 2B, 3A and 3B can be readily used in glycosidation, preferably sialylation and fucosylation reactions, such as sialylation reactions. Promoters can be selected, as in case of other glycosyl phosphites, from the group of Lewis acids like TMSOTf, BF₃.OEt₂, NIS, TfOH, LiClO₄, iodine, montmorillonite, Tf₂NH, ZnCl₂, Tf₂O or mixture thereof. The reaction runs in aprotic solvent, preferably in dichloromethane, THF, toluene, acetonitrile or in mixtures thereof, more preferably in dichloromethane/THF mixture, at temperatures between −78-0° C., preferably between −15 and −25° C.

Synthesis of oligosaccharides including sialooligosaccharides and fucooligosaccharides, such as sialooligosaccharides, generally follows multistep reaction sequence consisting of orthogonal protection—glycosylation—selective deprotection cascades until the target is reached. In large scale or industrial realization adjusting technological time is among crucial concerns. The main drawback of the multistep procedures is the unavoidable chromatographic separation in order either to get the pure substance/intermediate or to obtain at least a mixture that is enriched in the target compound but still contains undesired derivatives. Although repeated chromatographic separation may results in the improvement of the purity, its high cost and relatively long technological time to handle the feed solution and the column packing, to carry out the separation and optionally to regenerate the packing, especially in large or industrial scale, can be disadvantageous and/or cumbersome.

Crystallization or recrystallization is one of the simplest and cheapest methods to isolate a product from a reaction mixture, separate it from contaminations and obtain pure substance. Isolation or purification that uses crystallization makes the whole technological process robust and cost-effective, thus it is advantageous and attractive compared to other procedures.

Compounds of general formulae 1, 2A, 2B, 3A and/or 3B excellent choice as general/sialyl/fucosyl donor for glycosylation/sialylation/fucosylation reaction. As they can be synthesized in simple way in crystalline form with high purity and possess remarkable shelf-life they have obviously advantageous usability compared to other phosphites.

Accordingly, the present application relates to the synthesis of an oligosaccharide, characterized in that the said synthesis comprises at least the step of: coupling a compound of general formula 1 as defined above with an acceptor of the formula B—OH, wherein B—OH means a mono-, di- or oligosaccharide in protected form.

The acceptor B—OH in suitably protected form means any derivatized or non-derivatized mono-, di- or oligosaccharide whose functional groups are protected except for the OH-group to be coupled. In exceptional cases the group B may contain 1 or 2 additional free hydroxyl groups, preferably whose reactivity is much diminished than of that to be coupled, e.g. due to steric hindrance. The unprotected OH-group or one of the OH-groups is preferably not anomeric OH. The protective groups on compound B—OH may be identical, similar or different to those present in the donor of general formula 1. If the glycosyl residue in group B differs from monosaccharide, it may represent a linear or branched structure, consisting of monosaccharide units that are attached to each other by interglycosidic linkages. The monosaccharides units in compounds B—OH can be selected from any 5-9 carbon atom containing sugars consisting of aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglycosamine, N-acetylmannosamine, N-acetylgalactosamine, etc.), uronic acids, ketoaldonic acids (e.g. sialic acid) and like. The protective groups can be the commonly used ones in organic/carbohydrate chemistry, such groupings have been mentioned above.

The coupling reaction can be carried out in the presence of promoters can be selected, as in case of other glycosyl phosphites, from the group of Lewis acids like TMSOTf, BF₃.OEt₂, NIS, TfOH, LiClO₄, iodine, montmorillonite, Tf₂NH, ZnCl₂, Tf₂O or mixture thereof. The reaction runs in aprotic solvent, preferably in dichloromethane, THF, toluene, acetonitrile or in mixtures thereof, more preferably in dichloromethane/THF mixture, at temperatures between −78-0° C., preferably between −15 and −25° C.

The coupled product of formula A-O—B can thus be obtained which is a protected di- or oligosaccharide. If di- or oligosaccharide of formula A-O—B has been targeted to synthesize, the compounds is then subjected to remove the protective groups present. Removal of the masking groups can be carried out in one step or more consecutive steps. It is within the skilled person competence to select the appropriate reagent(s) and condition(s) for this purpose. For general considerations it is referred to the following books and reviews: P. G. M. Wuts and T. W. Greene: Protective Groups in Organic Synthesis, John Wiley & Sons (2007); S. Hanessian: Preparative Carbohydrate Chemistry Marcel Dekker (1997); Chemical Synthesis of Glycosides and Glycomimetics in: Carbohydrates in Chemistry and Biology (Eds.: B. Ernst, G. W. Hart, P. Sinaÿ) Part I, Vol. 1, Wiley (2000).

In case of need, compounds of formula A-O—B can be deprotected selectively to set free OH-group(s) for further manipulations such as glycosylation or derivatization. The skilled person is capable of choosing the appropriate reagent(s) and condition(s) in order to deprotect one or more functional groups while the other groups remain intact. In a certain phase of the reaction sequence the so obtained oligosaccharide can then be deprotected (vide supra).

In preferred embodiment compounds of general formula 1 are in fact compounds of general formulae 2A or 2B, such as compounds of general formula 2A, as defined above. In this particular case a compound of formula B—OH is sialylated or fucosylated giving rise to a compound of general formulae 4A or 4B, such as of general formula 4A,

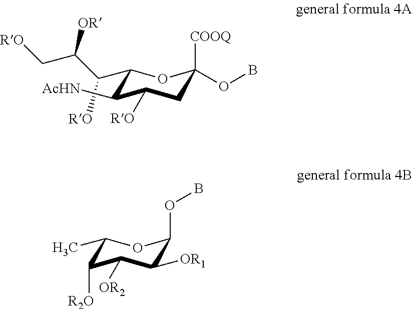

wherein R′ is optionally substituted acyl, Q is optionally substituted alkyl, R₁ is a group removable by hydrogenolysis or optionally substituted acyl, and R₂ is a group removable by hydrogenolysis, optionally substituted acyl or two R₂ groups together form a moiety

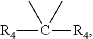

wherein R₃ and R₄, independently, are alkyl or phenyl, or wherein groups R₃ and R₄ together with the carbon atom to which they are attached form cycloalkylidene, and B is a mono-, di- or oligosaccharide in suitably protected form as defined above. Compounds of general formulae 4A and 4B are protected/partially protected sialo- or fucooligosaccharides with α-glycosidic linkage.

A more preferred embodiment relates to the synthesis of compounds of general formulae 5A and 5B. such as of general formula 5A,

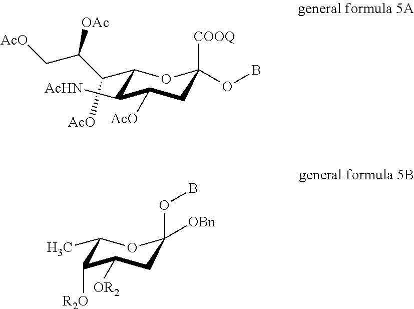

wherein Q is C₁₋₆ alkyl or benzyl, preferably methyl, R₂ is benzyl, acetyl or benzoyl optionally substituted with chloro, and B is a mono-, di- or oligosaccharide in protected form as defined above, using compounds of general formulae 3A and 3B, such as of general formula 3A, as defined above.

An even more preferred embodiment relates to the synthesis of a sialylated or fucosylated human milk oligosaccharide, such as a sialylated human milk oligosaccharide, characterized in that the said synthesis comprises at least the step of: coupling a compound of general formulae 3A or 3B, such as of general formula 3A, with an acceptor of the formula C—OH, wherein C—OH means a desialo or defuco human milk oligosaccharide in protected form.

Among sialo- and fucoglycoconjugates, sialylated or fucosylated human milk oligosaccharides like 3′-sialyllactose, 6′-sialyllactose, 2′-fucosyllactose, 3-fucosyllactose, 3′-sialyl-3-fucosyllactose, 2′,3-difucosyllactose, sialyllacto-N-tetraoses (LST a, LST b, LST c), sialyl-fucosyllacto-N-tetraoses (FLST a, FLST b, FLST c), lacto-N-fucopentaoses (LNFP I, LNFP II, LNFP III, LNFP V), lacto-N-difucohexaoses (LNDFH I, LNDFH II, LNDFH III), disialyllacto-N-tetraoses, sialyllacto-N-fucopentaoses, monosialyllacto-N-hexaoses, monosialyllacto-N-neohexaoses, monofucosyllacto-N-hexaoses, monofucosyllacto-N-neohexaoses, monofucosyl-monosialyllacto-N-hexaoses, monofucosyl-monosialyllacto-N-neohexaoses, monofucosyl-disialyllacto-N-hexaoses, monofucosyl-disialyllacto-N-neohexaoses, etc., such as 3′-sialyllactose, 6′-sialyllactose, 3′-sialyl-3-fucosyllactose, sialyllacto-N-tetraoses, sialyl-fucosyllacto-N-tetraoses, disialyllacto-N-tetraose, sialyllacto-N-fucopentaose, monosialyllacto-N-hexaose, monofucosyl-monosialyllacto-N-hexaose, monofucosyl-monosialyllacto-N-neohexaose, monofucosyl-disialyllacto-N-neohexaose, etc., are of great importance which is directly linked to their unique biological activities such as antibacterial, antiviral, immune system and cognitive development enhancing activities. Sialylated and fucosylated human milk oligosaccharides, such as sialylated human milk oligosaccharides, are found to act as prebiotics in the human intestinal system helping to develop and maintain the intestinal flora. Furthermore they have also proved to be anti-inflammatory, and therefore these compounds are attractive components in the nutritional industry for the production of, for example, infant formulas, infant cereals, clinical infant nutritional products, toddler formulas, or as dietary supplements or health functional food for children, adults, elderly or lactating women, both as synthetically composed and naturally occurring compounds and salts thereof. Likewise, the compounds are also of interest in the medicinal industry for the production of therapeutics. In the human milk oligosaccharides the sialic acid residue is always linked to the terminal 3-O— and/or 6-O— position(s) of D-galactose and/or to the 6-O positions of N-acetylglucosamine building blocks via α-glycosidic linkage, whereas the fucose moiety is attached to the galactose of the lacto-N-biosyl group with 1-2 interglycosidic linkage and/or to the N-acetyl-glucosamine of the lacto-N-biosyl group with 1-4 interglycosidic linkage and/or to the N-acetyl-glucosamine of the N-acetyl-lactosaminyl group with 1-3 interglycosidic linkage, and/or to the galactose of the lactosyl group with 1-2 interglycosidic linkage and/or to the glucose of the lactosyl group with 1-3 interglycosidic linkage, in all the cases via α-glycosidic bond.

Desialo- and defuco-human milk oligosaccharides in suitably protected form as compound C—OH intends to mean di- or oligosaccharides such as lactose, 3-fucosyllactose, 3′-sialyllactose, 2′-fucosyllactose, lacto-N-tetraose, lacto-N-neotetraose, fucosyllacto-N-tetraoses (lacto-N-fucopentaoses), monosialyllacto-N-tetraoses (LST a, LST b, LST c), lacto-N-hexaoses, lacto-N-neohexaoses, monofucosyl-lacto-N-hexaoses, monofucosyl-lacto-N-neohexaoses, monosialyl-lacto-N-hexaoses, monosialyl-lacto-N-neohexaoses, monofucosyl-monosialyllacto-N-neohexaoses, monofucosyl-monosialyllacto-N-hexaoses, etc., that is derivatives of natural sialylated and/or fucosylated human milk oligosaccharides (see above) in which at least one sialyl or fucosyl residue is not present. Desialo-human milk oligosaccharide in suitably protected form as compound C—OH intends to mean di- or oligosaccharides such as lactose, 3-fucosyllactose, lacto-N-tetraose, fucosyllacto-N-tetraose, monosialyllacto-N-tetraose, lacto-N-fucopentaose, lacto-N-hexaose, monofucosyl-lacto-N-hexaose, monofucosyl-lacto-N-neohexaose, monofucosyl-monosialyllacto-N-neohexaose, etc., that is sialylated human milk oligosaccharides (see above) in which at least one sialyl residue is not present. The functional groups in compounds C—OH are protected except for the OH-group to be coupled. In exceptional cases they may contain 1 or 2 additional free hydroxyl groups, preferably whose reactivity is much diminished than of that to be coupled, e.g. due to steric hindrance. The protective groups on compound C—OH may be identical, similar or different to those present in the donor of general formulae 1, 2A, 2B, 3A or 3B. Such masking groups are mentioned above.

The coupled products so obtained are in fact protected/partially protected sialylated and/or fucosylated human milk oligosaccharides, such as protected/partially protected sialylated human milk oligosaccharides. They can be subjected to remove the protective groups present. Removal of the masking groups can be carried out in one step or more consecutive steps. It is within the skilled person competence to select the appropriate reagent(s) and condition(s) for this purpose. Sialylated and fucosylated human milk oligosaccharides can be isolated from the reaction mixture using conventional work-up procedures both in solid form such as amorphous/freeze dried/spray dried or crystalline form and in liquid form as syrup or concentrated aqueous solution.

In an especially preferred embodiment the sialylated or fucosylated human milk oligosaccharide is selected from 6′-sialyllactose, 3′-sialyllactose, 2′-fucosyllactose, 3-fucosyllactose, 2′,3-difucosyllactose, 3′-sialyl-3-fucosyllactose, sialyllacto-N-tetraoses (LST a, LST b, LST c), sialyl-fucosyllacto-N-tetraoses (FLST a, FLST b, FLST c), lacto-N-fucopentaoses (LNFP I, LNFP II, LNFP III, LNFP V), lacto-N-difucohexaoses (LNDFH I, LNDFH II, LNDFH III) and disialyllacto-N-tetraose, more preferably from 6′-sialyllactose, 3′-sialyllactose, 2′-fucosyllactose, 3-fucosyllactose, 2′,3-difucosyllactose and 3′-sialyl-3-fucosyllactose.

In another especially preferred embodiment the sialylated human milk oligosaccharide is selected from 6′-sialyllactose, 3′-sialyllactose, 3′-sialyl-3-fucosyllactose, sialyllacto-N-tetraoses, sialyl-fucosyllacto-N-tetraoses and disialyllacto-N-tetraose, more preferably from 6′-sialyllactose and 3′-sialyllactose.

According to another embodiment, the synthesis of a mixture of sialylated human milk oligosaccharides is performed, characterized in that the said synthesis comprises at least the step of: coupling a compound of general formula 3A with a mixture comprising two or more desialo-human milk oligosaccharide in protected form. Similarly, the synthesis of a mixture of fucosylated human milk oligosaccharides is performed, characterized in that the said synthesis comprises at least the step of: coupling a compound of general formula 3B with a mixture comprising two or more defuco-human milk oligosaccharide in protected form.

The mixture of coupled products so obtained is in fact a mixture of protected sialylated human milk oligosaccharides or a mixture of protected fucosylated human milk oligosaccharides. They can be subjected to remove the protective groups present. Removal of the masking groups can be carried out in one step or more consecutive steps, such as by catalytic hydrogenolysis. It is within the skilled person competence to select the appropriate reagent(s) and condition(s) for this purpose. The mixture of sialylated human milk oligosaccharides or the mixture of protected fucosylated human milk oligosaccharides can be isolated from the reaction mixture using conventional work-up procedures both in solid form such as amorphous/freeze dried/spray dried or crystalline form and in liquid form as syrup or concentrated aqueous solution.

According to a preferred realization the mixture of sialylated human milk oligosaccharides comprises at least two sialylated human milk oligosaccharides selected from 6′-sialyllactose, 3′-sialyllactose, 3′-sialyl-3-fucosyllactose, sialyllacto-N-tetraoses (LST a, LST b, LST c), sialyl-fucosyllacto-N-tetraoses (FLST a, FLST b, FLST c) and disialyllacto-N-tetraose, whereas the mixture of protected fucosylated human milk oligosaccharides comprises at least two fucosylated human milk oligosaccharides selected from 2′-fucosyllactose, 3-fucosyllactose, 2′,3-difucosyllactose, 3′-sialyl-3-fucosyllactose, sialyl-fucosyllacto-N-tetraoses (FLST a, FLST b, FLST c), lacto-N-fucopentaoses (LNFP I, LNFP II, LNFP III, LNFP V) and lacto-N-difucohexaoses (LNDFH I, LNDFH II, LNDFH III). According to another preferred realization the mixture of sialylated human milk oligosaccharides comprises at least two sialylated human milk oligosaccharides selected from 6′-sialyllactose, 3′-sialyllactose, 3′-sialyl-3-fucosyllactose, sialyllacto-N-tetraoses, sialyl-fucosyllacto-N-tetraoses and disialyllacto-N-tetraose.

Synthetic usefulness of diphenyl-phosphite donors according to the present invention is demonstrated by the synthesis of 6′-sialyllactose and 2′-fucosyllactose, two prominent members of the human milk oligosaccharides. Other features of the invention will become apparent in the course of the following descriptions of exemplary embodiments which are given for illustration of the invention and are not to be limiting thereof.

### Examples

### Donors

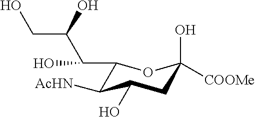

Method A: A mixture of anhydrous sialic acid (100 g, 323 mmol) and dried Amberlite IR-120 (H⁺) ion exchange resin (100 g) in MeOH (1500 mL) was stirred for 15 hours at RT. The ion exchange resin was filtered off and washed with MeOH (2×100 mL). The washes were combined with the filtrate and concentrated to 300 mL. The concentrated residue crystallized upon seeding at RT. The crystals were collected by filtration giving 73.8 g (71%) sialic acid methyl ester. The mother liquor was concentrated (10.5 g) and recrystallized from MeOH (30 mL) to yield 7.4 g (7%) sialic acid methyl ester. Total yield 81.2 g (78%).

Method B: To a suspension of anhydrous sialic acid (100 g, 323 mmol) in MeOH (1200 ml) 8% HCl in MeOH (50 ml) was added and the reaction mixture was stirred for 6 hours at RT. The reaction mixture was neutralized with triethylamine (15 ml) and the clear solution was concentrated to 270 mL. The concentrated residue crystallized upon seeding at RT for 2 hours. The solid was collected by filtration yielding 104.9 g (100%). ¹H NMR (D₂O) δ in ppm: 1.89 (dd, 1H, J=13.0 Hz, J=11.4 Hz); 2.03 (s, 3H); 2.28 (dd, 1H, J=13.0 Hz, J=4.7 Hz); 3.52 (dd, 1H, J=8.9 Hz, J=3.0 Hz); 3.59 (dd, 1H, J=11.6 Hz, J=6.1 Hz); 3.71 (ddd, J=8.9 Hz, J=2.5 Hz, J=11.6 Hz); 3.82 (dd, 1H, J=11.6 Hz, J=2.5 Hz); 3.82 (s, 3H); 3.90 (dd, 1H, J=10.1 Hz, J=10.0 Hz); 3.98-4.10 (m, 2H, H-6, H-4).

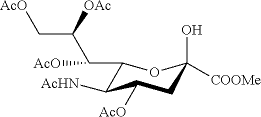

Method A: A suspension of sialic acid methyl ester (50 g, 155 mmol) and acetic anhydride (73 ml, 775 mmol) in DCM (175 mL) was stirred at RT and 70% perchloric acid (1 mL) was then added dropwise within 30 minutes. During the addition the temperature of the mixture increased until reflux. The reaction mixture was stirred at reflux for 2.5 h, and after this time MeOH (7.5 ml, 185 mmol) was added dropwise and the reaction mixture was stirred for a further hour at RT. The reaction mixture was diluted with DCM (175 mL) and washed with water (3×50 mL). The combined water phases were extracted with DCM (2×100 mL). The combined organic phases were washed with saturated NaHCO₃ (2×100 mL) and evaporated. The residue (59.6 g) was dissolved in iBuOAc at 50° C. and the mixture was cooled down to RT and let overnight complete the crystallization. The solid was collected by filtration yielding 39.2 g (52%) of tetraacetyl sialic acid methyl ester.

Method B: To a mixture of sialic acid methyl ester (60.8 g, 188 mmol) and acetic anhydride (89 ml, 940 mmol) in DCM (220 mL), perchloric acid 70% (1.22 mL) was added dropwise within 30 minutes. During the addition the temperature of the mixture increased until reflux (47° C.). The reaction mixture is stirred at reflux for 2.5 h. Subsequently, MeOH (9.2 mL, 225 mmol) was added dropwise and the reaction mixture was stirred for one additional hour at RT. The clear solution was added dropwise to a suspension of Na₂CO₃ (60.8 g; 573 mmol) in DCM and the mixture was stirred at room temperature for 2 hours. The remaining solid was removed by filtration and was washed with DCM (2×50 mL). The combined DCM phase was concentrated to 150 ml, ⁱBuOAc (150 mL) was added and the remaining DCM was removed and let crystallize overnight. The crystals were collected by filtration yielding 65 g (71%) of tetraacetyl sialic acid methyl ester. ¹H NMR(C₆D₆) δ in ppm: 1.60, 1.63, 1.70, 1.85, 1.92 (5 s, 15H); 2.19 (dd, 1H, J=12.8 Hz, J=5.7 Hz); 2.25 (ddd, 1H, J=12.8 Hz, J=10.8 Hz); 3.28 (s, 3H); 4.23 (dd, 1H, J=12.4 Hz, J=7.6 Hz); 4.26 (dd, 1H); 4.54 (ddd, 1H, J=10.8 Hz); 4.78 (d, 1H, J=10.2 Hz); 5.02 (dd, 1H, J=12.4 Hz, J=2.0 Hz); 5.26 (ddd, 1H, J=10.8 Hz, J=5.7 Hz, J=10.5 Hz); 5.61 (ddd, 1H, J=2.0 Hz, J=7.6 Hz); 5.64 (dd, 1H, J=2.3 Hz, J=4.2 Hz). ¹H NMR (CDCl₃) δ in ppm: 1.91, 2.02, 2.03, 2.11, 2.15 (5 s, 15H); 2.19 (dd, 1H, J=12.8 Hz, J=11.4 Hz); 3.26 (ddd, 1H, J=12.8 Hz, J=5.4 Hz); 3.86 (s, 3H); 4.03 (dd, 1H, J=12.4 Hz, J=7.5 Hz); 4.13-4.21 (m, 2H); 4.51 (dd, 1H, J=12.4 Hz, J=2.4 Hz); 5.22 (ddd, 1H, J=11.4 Hz, J=5.4 Hz, J=9.5 Hz); 5.25 (ddd, 1H, J=2.4 Hz, J=7.5 Hz, J=5.6 Hz); 5.36 (dd, 1H, J=1.5 Hz, J=5.6 Hz); 5.71 (m, 1H). ¹³C NMR: 20.65, 20.75, 20.93, 22.93, 36.11, 49.04, 53.18, 62.50, 68.3, 69.12, 71.32, 72.07, 94.84, 168.93, 170.12, 170.30, 170.72, 171.04, 171.43.

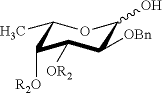

Partially protected fucose derivatives (R₂ means benzoyl or 4-chlorobenzoyl) with free anomeric OH were prepared analogously to Eisele et al. Carbohydr. Res. 306, 81 (1998).

### General Procedure of Preparing Glycosyl Phosphites:

DCM, Et₃N (5.5 eq.) and imidazole (4.5 eq.) were placed into the flask and the suspension was cooled to 5° C. PCl₃ (1.5 eq.) was then added and the reaction mixture was stirred for 1 h at 5° C. One equivalent of 1-OH-sugar derivative (N-acetyl neuraminic acid methyl ester tetra-O-acetate, or 2-O-benzyl-3,4-di-O-benzoyl-L-fucose, or 2-O-benzyl-3,4-di-O-(4-chlorobenzoyl)-L-fucose) in DCM was added and the temperature allowed to warm up to 20° C. The mixture was stirred for 3 h at r.t. followed by the addition of the phenol derivative (3.5 eq.). The mixture was stirred for 1 h. The base was neutralized by addition of 2 N HCl solution and the mixture was stirred vigorously for 15 min. The phases were separated. The organic phase was washed with NaHCO₃ solution and with water. The organic phase was concentrated and in case of sialic acid derivatives the product was crystallized, or in case of the fucose derivatives the product was isolated after chromatography (yield: 70-80%).

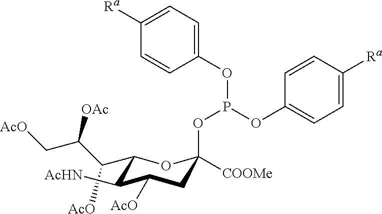

R^{a}=methyl: mp: 128.3-130.8° C.; ¹H NMR (CDCl₃) δ (ppm): 1.84 (s, 3H); 1.90 (m, 3H); 2.00 (s, 3H); 2.04 (s, 3H); 2.10 (m, 4H); 2.31 (s, 6H); 2.47 (dd, 1H, J=4.9 Hz, J=13.1 Hz); 3.75 (s, 3H); 3.9 (dd, 1H, J=2.3 Hz, J=10.7 Hz); 4.1 (m, 2H); 4.55 (dd, 1H, J=12.3 Hz, J=2.4 Hz); 4.87 (d, 1H, J=10.3 Hz); 5.1 (m, 2H); 5.27 (m, 1H); 6.97-7.2 (m, 8H). ¹³C NMR (CDCl₃) δ (ppm): 20.93, 20.96, 20.99, 21.09, 21.30, 23.37, 38.16, 48.93, 53.62, 62.75, 68.25, 68.64, 72.25, 72.77, 98.84, 98.87, 121.05, 121.14, 130.41, 130.50, 133.98, 134.08, 149.33, 149.35, 167.13, 170.25, 170.29, 170.60, 170.81, 170.97.

R^{a}=methoxy: mp: 124.8-126.7° C.; ¹H NMR (CDCl₃) δ (ppm): 1.84 (s, 3H); 1.88 (m, 3H); 2.00 (s, 3H); 2.01-2.07 (m, 4H); 2.09 (s, 3H); 2.44 (dd, 1H, J=4.9 Hz, J=13 Hz); 3.64 (dd, 1H, J=2.5 Hz, J=10.7 Hz); 3.77 (m, 9H); 4.07 (m, 2H); 4.55 (dd, 1H, J=12.3 Hz, J=2.3 Hz); 4.8 (d, 1H, J=10.4 Hz); 5.01 (dd, 1H, J=10.9 Hz, J=4.9 Hz); 5.08 (m, 1H); 5.22 (t, 1H, J=2.8 Hz); 6.9 (m, 4H); 7.1 (m, 4H). ¹³C NMR (CDCl₃) δ (ppm): 20.93, 20.98, 21.09, 21.30, 23.15, 38.13, 48.72, 53.71, 55.84, 62.79, 68.33, 68.52, 72.50, 72.61, 98.93, 114.95, 115.07, 122.37, 122.46, 122.62, 122.62, 144.98, 144.05, 144.08, 154.41, 167.21, 170.30, 170.33, 170.67, 170.80, 170.93.

R^{a}═H: mp: 122.3-123.9° C.; ¹H NMR (CDCl₃) δ (ppm): 1.84 (s, 3H); 1.89 (m, 3H); 2.00 (s, 3H); 2.04 (s, 3H); 2.10 (m, 4H); 2.47 (dd, 1H, J=4.9 Hz, J=13.1 Hz); 3.75 (s, 3H); 3.87 (dd, 1H, J=2.4 Hz, J=10.7 Hz); 4.1 (m, 2H); 4.55 (dd, 1H, J=12.3 Hz, J=2.4 Hz); 4.80 (d, 1H, J=10.2 Hz); 5.05 (dd, 1H, J=4.9 Hz, J=10.9 Hz); 5.12 (m, 1H); 5.26 (dd, 1H, J=2.5 Hz, J=3.5 Hz); 7.1-7.25 (m, 6H); 7.31-7.41 (m, 4H). ¹³C NMR (CDCl₃) δ (ppm): 20.92, 20.98, 21.09, 21.30, 23.38, 38.12, 48.76, 53.71, 62.75, 68.29, 68.57, 72.31, 72.86, 99.03, 99.06, 121.33, 121.42, 121.45, 121.54, 124.45, 124.50, 130.05, 130.14, 151.70, 167.12, 170.25, 170.27, 170.65, 170.80, 170.97.

R^{a}=Br: ¹H NMR (CDCl₃) δ (ppm): 1.88 (s, 6H); 2.01 (s, 3H); 2.03 (s, 3H); 2.09 (m, 4H); 2.44 (dd, 1H, J=4.9 Hz, J=13.2 Hz); 3.78 (s, 3H); 3.85 (dd, 1H, J=2.6 Hz, J=10.6 Hz); 3.96-4.21 (m, 2H); 4.51 (dd, 1H, J=12.3 Hz, J=2.3 Hz); 4.94 (d, 1H, J=10.2 Hz); 5.05 (m, 2H); 5.26 (m, 1H); 7.04 (m, 4H); 7.47 (m, 4H). ¹³C NMR (CDCl₃) δ (ppm): 20.90, 20.96, 21.05, 21.28, 23.43, 38.15, 48.88, 53.92, 62.65, 68.32, 68.39, 72.32, 73.17, 99.40, 117.18, 123.01, 123.10, 123.25, 123.33, 133.02, 133.09, 150.59, 150.62, 167.09, 170.23, 170.51, 170.72, 170.97.

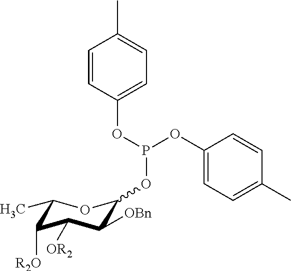

R₂=4-chlorobenzoyl, α-anomer: ¹H NMR (CDCl₃) δ (ppm): 1.12 (s, 3H, J=6.0 Hz), 2.33 (s, 6H), 4.16 (dd, 1H, J=3.0 12.0 Hz), 4.45-4.52 (m, 1H), 4.63 (d, 1H, J=12.0 Hz), 4.69 (d, 1H, J=12.0 Hz), 5.65-5.67 (m, 1H), 5.79 (dd, 1H, J=3.0 12.0 Hz), 6.15 (dd, 1H, J=3.0 9.0 Hz), 7.13-7.92 (m, 21H). ¹³C NMR (CDCl₃) δ (ppm): 15.8, 20.6 (2C), 66.2, 70.5, 72.2, 72.4, 72.7 (d, J=4.0 Hz), 91.9 (d, J=9.0 Hz), 120.2 (d, J=6.8 Hz), 120.4 (d, J=3.8 Hz), 127.8-130.9, 133.4, 133.4, 137.2, 139.4, 139.8, 149.4-149.6 (2C), 164.5, 164.9.

R₂=4-chlorobenzoyl, β-anomer: ¹H NMR (CDCl₃) δ (ppm): 1.18 (d, 3H, J=6.0 Hz), 2.21 (s, 3H), 2.22 (s, 3H), 3.84-4.00 (m, 2H), 4.49 (d, 1H, J=12.0 Hz), 4.66 (d, 1H, J=12.0 Hz), 5.28-5.38 (m, 2H), 5.49 (s, 1H), 6.97-7.88 (m, 21H). ¹³C NMR (CDCl₃) δ (ppm): 16.1, 20.7 (2C), 69.9, 71.4, 72.4, 73.1, 74.6, 76.3 (d, J=4.5 Hz), 97.4 (d, J=12.8 Hz), 120.2 (d, J=7.5 Hz), 120.7 (d, J=6.0 Hz), 127.6-131.2, 133.5, 133.6, 137.5, 139.6, 139.9, 164.5, 165.0.

R₂=benzoyl, α-anomer: ¹H NMR (CDCl₃) δ (ppm): 1.10 (d, 3H, J=6.0 Hz), 2.31 (s, 6H), 4.19 (dd, 1H, J=3.0 12.0 Hz), 4.43-4.49 (m, 1H), 4.63 (d, 1H, J=12.0 Hz), 4.67 (d, 1H, J=12.0 Hz), 5.67 (dd, 1H, J=1.2 3.3 Hz), 5.79 (dd, 1H, J=3.3 10.5 Hz), 6.10 (dd, 1H, J=3.6 8.4 Hz), 7.08-7.98 (m, 23H). ¹³C NMR (CDCl₃) δ (ppm): 15.9, 20.7 (2C), 66.4, 70.4, 72.0, 72.6, 73.0 (d, J=3.8 Hz), 92.2 (d, J=9.0 Hz), 120.3 (d, J=7.5 Hz), 120.4 (d, J=6.8 Hz), 127.7-128.5, 129.5-130.0, 133.0, 133.2, 133.4, 133.4, 137.4, 149.5, 149.6, 165.8, 165.9.

R₂=benzoyl, β-anomer: ¹H NMR (CDCl₃) δ (ppm): 1.17 (d, 3H, J=6.0 Hz), 2.18 (s, 6H), 3.89-3.97 (m, 2H), 4.51 (d, 1H, J=12.0 Hz), 4.65 (d, 1H, J=12.0 Hz), 5.32-5.37 (m, 2H), 5.51 (dd, 1H, J=0.9 3.6 Hz), 6.95-7.96 (m, 23H). ¹³C NMR (CDCl₃) δ (ppm): 16.1, 20.4 (2C), 70.1, 71.3, 73.2, 74.7, 76.6, 97.4 (d, J=12.8 Hz), 120.2 (d, J=6.8 Hz), 120.8 (d, J=6.0 Hz), 127.5-128.5, 129.2-130.1, 133.2, 133.4, 133.5, 133.6, 137.5, 149.2 (d, J=3.2 Hz), 149.5 (d, J=5.3 Hz), 165.8, 166.1.

### Acceptors

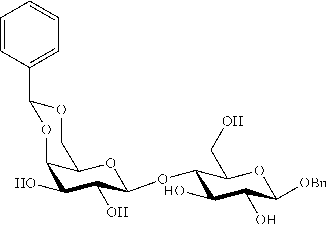

To a suspension of 6-benzyllactoside (700 g, 1.6 mol) in DMF (5 L) was added benzaldehyde dimethylacetal (389.5 mL, 2.6 mmol, 1.6 eq.) and p-TsOH.H₂O (31.5 g, 0.17 mmol, 0.1 eq.). The reaction mixture was then heated to 40-44° C. for approximately 22 h, after which a white suspension was obtained. It was cooled to ice bath temperature, ⁱPr₂O (4 L) was added and the resulting suspension was further stirred for 1½ h at this temperature. This mixture was filtrated and the white solid obtained was washed/suspended with ⁱPr₂O (2×1 L). After drying 702 g were obtained of the wanted product as a white free solid. The resulting mother liquor was allowed to stand at room temperature for approximately 3 days, during which time more white solid appeared. This was filtrated and washed/suspended with ⁱPr₂O (2×150 mL). Obtained further 25 g of product. Combined amount: 727 g (86%). ¹H NMR (CD₃OD) δ in ppm: 3.3-3.45 (m, 2H); 3.5-3.7 (m, 5H); 3.95 (m, 2H); 4.15 (m, 3H); 4.40 (d, 1H, J=7.8 Hz); 4.5 (d, 1H, J=7.5 Hz); 4.65 (d, 1H, J=11.8 Hz); 4.9 (d, 1H, J=11.8 Hz); 5.55 (s, 1H); 7.25 (m, 5H). ¹³C NMR: 61.75, 68.33, 70.17, 71.78, 71.86, 73.52, 74.89, 76.33, 77.35, 80.02; 102.3, 103.22, 104.87; 128.8-139.03.

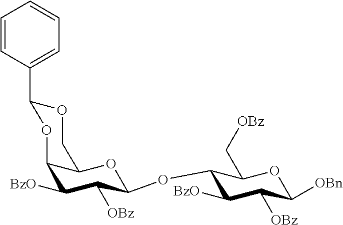

To an ice bath cooled solution of benzylidene-β-benzyllactoside (example 9, 6.2 g, 11.9 mmol) in pyridine (40 mL), BzCl (13.8 mL) was added dropwise. The reaction mixture stirred 30 min at this temperature, and overnight at RT. The reaction was quenched with MeOH and the solvent was evaporated in vacuo. The residue was dissolved in DCM and washed with water, 1N HCl, water, NaHCO₃, and brine. The organic phase was dried over Na₂SO₄ and the solvent was removed in vacuo. The solid obtained was recrystallized form EtOAc/Hexane to yield 9.1 g (73%) of a white pure solid. Mp.: 162-164° C. ¹H NMR (CDCl₃) δ (ppm): 2.95 (m, 1H); 3.58 (m, 1H); 3.78 (m, 2H); 4.25 (m, 2H); 4.40 (dd, 1H, J=4.3 Hz, J=12.1 Hz); 4.55 (d, 1H, 12.5 Hz); 4.65 (m, 1H); 4.70 (d, 1H, J=7.7 Hz); 4.78 (d, 1H, 12.5 Hz); 4.85 (d, 1H, J=7.9 Hz); 5.15 (dd, 1H, J=3.4 Hz, J=10.4 Hz); 5.30 (s, 1H); 5.40 (dd, 1H, J=7.8 Hz, J=9.2 Hz); 5.75 (m, 2H). 7.05-8.05 (m, 35H).

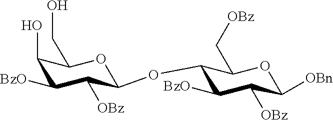

To a solution of the pentabenzoyl derivative (example 14, 22.0 g, 21 mmol) in DCM (140 mL) MeOH (20 mL) mixture TsOH monohydrate (1.6 g, 0.4 eq) was added, and the reaction mixture stirred for 2 days at 40° C. After this time a saturated solution of NaHCO₃ was added and the mixture stirred for 15 min. The phases were separated and the organic one was washed with water and brine; after drying over Na₂SO₄. The solvent was evaporated in vacuo and the solid obtained was suspended in EtOAc (200 mL) and the slurry stirred overnight, after filtration and drying 14.5 g (74%) were obtained. Mp.: 220-222.5° C. ¹H NMR (CDCl₃): 2.95 (m, 1H); 3.35 (m, 3H); 3.80 (m, 1H); 4.20 (m, 2H); 4.40 (dd, 1H, J=5 Hz, J=11.9 Hz); 4.5-4.9 (m, 5H); 5.07 (dd, 1H; J=3.1 Hz, 10.4 Hz); 5.45 (dd, 1H, J=7.7 Hz, J=9.4 Hz); 5.70 (m, 2H), 7.0-8.1 (m, 30H).

### Analogously Prepared:

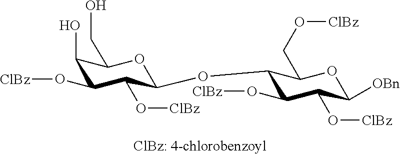

¹H NMR (CDCl₃): 2.95 (m, 1H); 3.40 (m, 3H); 3.85 (m, 1H); 4.15 (m, 2H); 4.40 (dd, 1H, J=5 Hz, J=11.9 Hz); 4.5-4.8 (m, 5H); 5.15 (dd, 1H; J=3.1 Hz, 10.4 Hz); 5.40 (dd, 1H, J=7.7 Hz, J=9.4 Hz); 5.65 (m, 2H), 7.0-8.0 (m, 25H).

### Analogously Prepared:

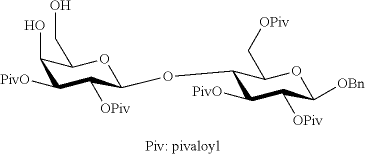

¹H NMR δ (CD₃OD) in ppm: 1.05-1.2 (4 s, 36H); 1.25 (s, 9H); 3.4 (m, 2H); 3.8 (m, 1H); 3.95 (m, 2H); 4.1 (m, 1H); 4.2 (dd, 1H, J=11.8 Hz, J=5.5 Hz); 4.55 (m, 4H); 4.8 (d, 1H, J=12.5 Hz); 4.9 (dd, 1H); 5.0 (dd, 1H); 5.2 (m, 2H); 7.2-7.4 (m, 5H).

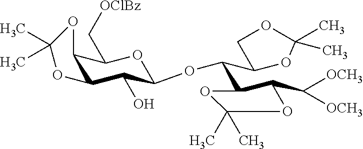

4-O-(6-O-(4-chlorobenzoyl)-3,4-O-isopropylidene-β-D-galactopyranosyl)-2,3:5,6-di-O-isopropylidene-D-glucose dimethyl acetal was prepared according to WO 2010/115935.

### Glycosylation: Sialylation

To a −20° C. cooled suspension of the donor (1.6 eq) and acceptor (1.0 eq) in DCM/THF triflic acid (0.16 eq) was added. The reaction mixture stirred for 3 h at −20° C. A solution of aqueous NaHCO₃ (was added to neutralize the acid. The phases were separated and the organic phase was washed with water and with water/brine mixture. The organic solvent was evaporated giving rise to the product as syrup.

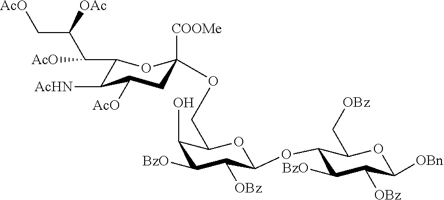

¹H NMR (CDCl₃) δ (ppm): 1.7 (s, 3H); 1.85 (s, 3H); 2.05 (m, 7H); 2.15 (s, 3H); 2.35 (dd, 1H, J=4.7 Hz, J=12.6 Hz); 3.07 (m, 1H); 3.3 (m, 1H); 3.4 (m, 1H); 3.7-3.85 (m, 4H); 3.9-4.1 (m, 4H); 4.17 (m, 1H); 4.4-4.6 (m, 3H); 4.6-4.9 (m, 4H); 5.05-5.35 (m, 4H); 5.45 (dd, 1H; J=7.8 Hz, J=9.6 Hz); 5.6-5.75 (m, 2H); 7.0-7.7 (m, 20H); 7.8-8.1 (m, 10H). ¹³C NMR (CDCl₃) δ (ppm): 20.55, 21.07, 21.12, 21.16, 21.24, 23.26, 35.73, 49.40, 53.26, 54.67, 60.24, 62.87, 62.97, 65.57, 67.88, 69.32, 70.15, 70.29, 70.56, 72.12, 72.86, 73.29, 73.59, 74.18, 74.51, 98.90, 99.02, 101.68, 127.88, 127.95, 128.05, 128.51, 128.59, 128.64, 129.15, 129.53, 129.60, 128.88, 130.07, 130.16, 133.33, 133.40, 133.69, 136.71, 165.22, 164.42, 165.50, 166.01, 166.07, 167.85, 170.47, 170.56, 170.65, 171.14, 171.39.

The same product was obtained using donors having R^{a}═H, methoxy and bromo.

### Analogously Prepared:

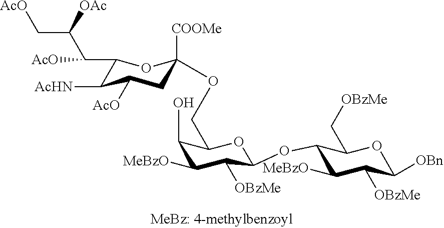

¹H NMR (CDCl₃) δ (ppm): 1.85 (s, 3H); 1.95 (m, 1H); 2.05, 2.07, 2.11, 2.15 (4 s, 12H); 2.3 (s, 3H); 2.35-2.45 (m, 13H); 3.0 (d, 1H, J=5.8 Hz); 3.25 (m, 1H); 3.32 (m, 1H); 3.45 (m, 1H); 3.75-3.85 (m, 4H); 3.95-4.05 (m, 3H); 4.1 (m, 1H); 4.25 (dd, 1H, J=9.15 Hz, J=9.55 Hz); 4.37 (dd, 1H, J=12 Hz, J=2.0 Hz); 4.42 (dd, 1H, J=5.1 Hz, J=12 Hz); 4.53 (d, 1H; J=12.5 Hz); 4.62 (dd, 1H; J=1.4 Hz, J=11.6 Hz); 4.67 (d, 1H, J=7.7 Hz); 4.76 (d, 1H, 12.6 Hz); 4.79-4.89 (m, 2H); 5.1 (dd, 1H, J=3 Hz, J=12.4 Hz); 5.2-5.35 (m, 3H); 5.37 (dd. 1H, J=7.8 Hz, J=9.3 Hz) 5.6-5.75 (m, 2H); 7.1-7.3 (m, 15H); 7.7-7.9 (m, 10H). ¹³C NMR (CDCl₃) δ (ppm): 20.75, 21.05, 21.14, 21.27, 21.66, 21.71, 21.85, 21.96, 23.42, 34.61, 36.39, 45.98, 49.59, 53.23, 54.66, 59.01, 60.73, 62.85, 65.85, 67.74, 69.14, 69.79, 69.96, 70.39, 72.16, 72.81, 73.19, 73.22, 73.74, 74.22, 76.71, 98.96 (2C), 101.58, 125.53, 126.83, 127.25, 127.90, 127.96, 128.46, 128.49, 129.15, 129.21, 129.26, 129.36, 129.88, 130.05, 130.12, 130.17, 136.83, 138.10, 143.72, 143.86, 143.96, 144.02, 144.06, 165.29, 165.54, 166.08, 167.84, 170.42, 170.48, 170.52, 171.18, 171.22.

### Analogously Prepared:

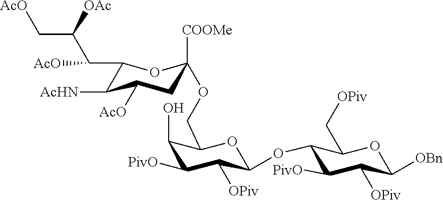

¹H NMR (CDCl₃) δ in ppm: 1.05-1.3 (m, 45H); 1.75-2.25 (m, 16H); 2.6 (dd, 1H, J=4.7 Hz, 12.6 Hz); 3.5-3.75 (m, 8H); 3.9-4.10 (m, 5H), 4.3 (m, 2H); 4.5 (m, 4H); 4.75-5 (m, 4H); 5.2 (m, 3H); 5.3 (m, 2H); 7.25 (m, 5H). ¹³C NMR (CDCl₃) δ (ppm): 21.02; 21.1; 21.32; 23.09; 23.45; 27.33; 27.38; 27.43; 27.51; 38.90; 39.08; 53.24; 62.34; 62.68; 62.70; 62.72; 66.44; 67.46; 68.87; 69.09; 69.4; 70.56; 71.78; 71.83; 73.00; 73.13; 73.43; 73.54; 73.88; 73.89; 99.26; 99.38; 99.92; 128.17; 128.57; 136.84; 168.09; 170.38; 170.41; 171.00; 171.22; 178.00.

### Analogously Prepared:

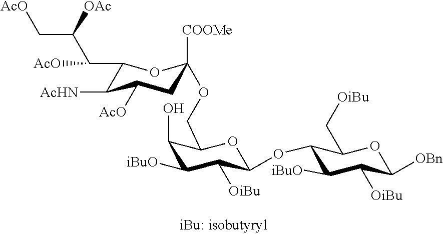

¹H NMR (CDCl₃) δ in ppm: 1.0-1.25 (m, 30H); 1.8-2.2 (m, 16H); 2.4-2.65 (m, 6H); 3.5-3.9 (m, 9H); 3.95-4.1 (m, 3H); 4.13-4.25 (m, 2H); 4.4-4.6 (m, 5H); 4.78-4.9 (m, 3H); 4.97 (dd, 1H, J=7.95 Hz, J=9.75 Hz); 5.13-5.25 (m, 3H); 5.3 (m, 2H); 7.2-7.35 (m, 5H). ¹³C NMR (CDCl₃) δ (ppm): 18.97, 19.08, 19.13, 19.21, 19.34, 19.58, 21.09, 21.30, 23.43, 34.13, 49.56, 50.29, 54.66, 62.09, 62.66, 66.37, 67.40, 68.87, 69.48, 70.69, 71.42, 71.96, 72.87, 73.02, 73.49, 75.35, 99.23, 99.35, 100.60, 127.99, 128.14, 128.58, 136.92, 168.05, 170.24, 170.37, 170.41, 171.04, 175.26, 175.71, 176.11, 176.29, 176.71.

### Glycosylation: Fucosylation

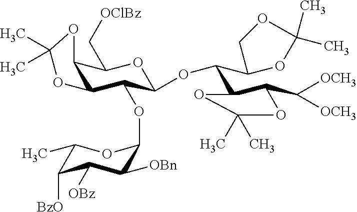

To a cooled solution of 2-O-benzyl-3,4-di-O-benzoyl-α-L-fucopyranosyl di-(4-methylphenyl) phosphite (1.2 eq) and 4-O-(6-O-(4-chlorobenzoyl)-3,4-O-isopropylidene-β-D-galactopyranosyl)-2,3:5,6-di-O-isopropylidene-D-glucose dimethyl acetal (1.0 eq) in DCM/2-methyltetrahydrofurane, triflic acid (0.16 eq.) was added. The reaction mixture stirred for 1 h at 0° C. and then a solution of aqueous NaHCO₃ was added to neutralize the acid. The phases were separated and the organic phase was washed with water and with water/brine mixture. The organic solvent was evaporated giving rise to the product as syrup. Characterization data were in accordance with those published in WO 2010/115935.

### Deprotection

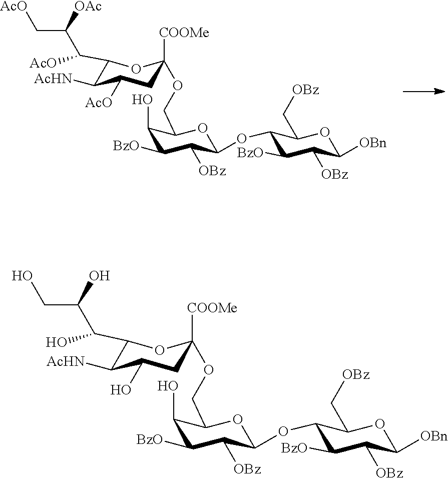

The syrup obtained above was diluted with MeOH, cooled to 5° C. and sulfuric acid was added dropwise. The reaction mixture was stirred for 48 h at this temperature and neutralized with Et₃N. MeOH was evaporated and the residue was dissolved in EtOAc, washed with water once and with 5/1 water brine. The organic phase was evaporated in vacuo and the residue was crystallized (yield: 50-60% for two steps). ¹H NMR (CD₃OD) δ (ppm): 1.5 (dd, 1H, J=11.7 Hz, J=12.6 Hz); 2.0 (s, 3H); 2.45 (dd, 1H, J=4.6 Hz, J=12.8 Hz); 3.2 (m, 1H); 3.4-3.7 (m, 7H); 3.7-3.85 (m, 5H); 3.95 (m, 1H); 4.1 (d, 1H, J=3.5 Hz); 4.25 (dd, 1H, J=9.2 Hz, J=9.5 Hz); 4.45-4.65 (m, 3H); 4.75 (d, 1H, J=12.2 Hz); 5.15 (dd, 1H, J=3.3 Hz, J=10.4 Hz); 5.35 (dd, 1H, J=8 Hz, J=9.7 Hz); 5.64 (dd, 1H, J=7.9 Hz, J=10.3 Hz); 5.69 (dd, 1H, J=9.2 Hz, J=9.4 Hz); 7.05-7.65 (m, 20H, 7.8-8.1 (m, 10H). ¹³C NMR (CD₃OD) δ (ppm): 21.62, 39.03, 52.37, 52.44, 60.04, 63.14, 65.25, 67.44, 68.41, 70.50, 70.87, 72.58, 73.24, 73.39, 73.66, 73.97, 74.79, 77.15, 127.73, 128.16, 128.27, 128.37, 128.63, 129.08, 129.41, 129.46, 129.49, 129.54, 129.59, 129.72, 129.81, 129.89, 133.18, 137.18, 165.63, 165.66, 165.95, 166.01, 166.22, 169.35, 173.99.

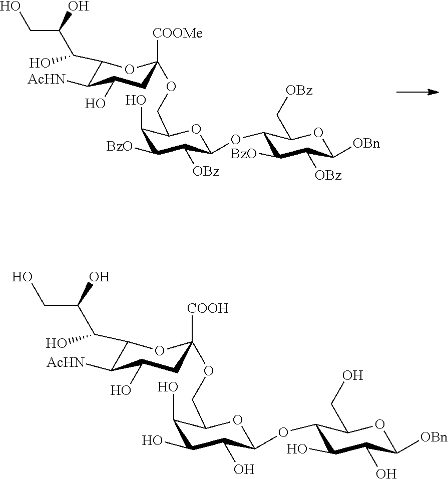

To a solution of the partially protected trisaccharide obtained above in 5 volumes of MeOH 1 M aqueous solution of NaOH was added slowly. The reaction mixture stirred overnight at RT and Amberlite IR 120 H⁺ was added to neutralize the base. The mixture was filtered and the solvent was evaporated in vacuo. The material was then dissolved in MeOH and EtOH, and TBME was added slowly. The precipitate was filtered off and washed twice with TBME. The white solid obtained was dissolved in 1 M NaOH and the reaction mixture was stirred for 6 h. IR-120 H⁺ was used to neutralize the base and the solvent was evaporated in vacuo. After coevaporation with EtOH (twice) the product was dissolved in MeOH and iPrOH was added slowly to give a solid in free acid form. ¹H NMR (CD₃OD) δ (ppm): 1.63 (t, 1H, J=11.9 Hz); 2.00 (s, 3H); 2.78 (dd, 1H, J=4.5 Hz, J=12.2 Hz); 3.28-3.49 (m, 4H); 3.50-3.79 (m, 9H); 3.80-3.97 (m, 5H); 4.02 (dd, 1H, J=7.5 Hz, J=10 Hz); 4.35 (m, 1H); 4.42 (d, 1H, J=7.8 Hz); 4.66 (d, 1H, J=11.7 Hz); 7.22-7.37 (m, 3H); 7.38-7.46 (m, 2H). ¹³C NMR (CD₃OD) δ (ppm): 21.55, 41.26, 52.70, 60.81, 63.21, 63.42, 68.56, 69.01, 69.30, 70.66, 71.15, 72.12, 73.08, 73.55, 73.78, 74.47, 75.28, 75.32, 80.03, 100.29, 101.89, 103.36, 127.36, 127.87, 128.01, 128.12, 137.72, 173.36, 173.88.

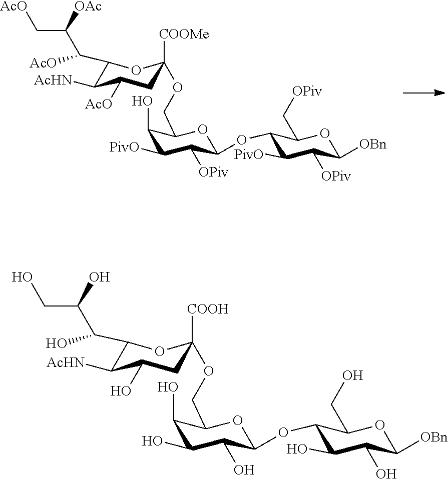

To a solution of a protected trisaccharide in MeOH NaOMe was added and the reaction mixture was stirred for 5 h at 45° C. The cooled solution was washed with heptane and acetone was added. The precipitate was filtered off and dissolved in 1 M NaOH and the reaction mixture was stirred for 6 h. IR-120 H⁺ was used to neutralize the base and the solvent was evaporated in vacuo. After co-evaporation with EtOH (twice) the product was dissolved in MeOH and iPrOH was added slowly to give a solid in free acid form.

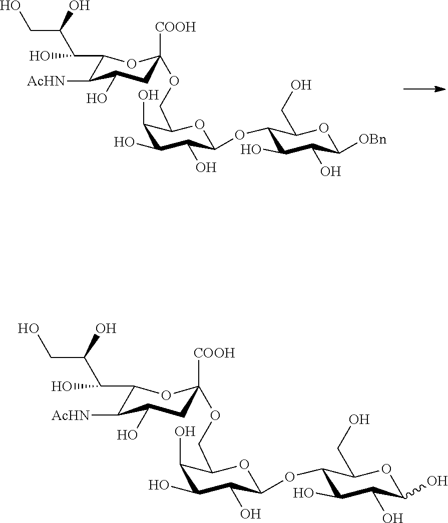

To a solution of 40 g of free acid in a mixture of methanol and water (250 mL+300 mL) 4 g of Pd/C (10%) were added. The reaction mixture was stirred 2 d at RT under H₂ pressure (balloon). The mixture was then filtered through a pad of Celite and the solvent was evaporated in vacuo. The residue was dissolved in 80 mL of H₂O and dropped to 1200 mL of EtOH. The slurry was filtrated, the solid was washed with EtOH, acetone and a mixture of 1/1 acetone/Et₂O. The solid was dried to give 35 g of 6′-sialyllactose. ¹H NMR (D₂O) (anomeric mixture of glucose 0.6/0.4 β/α): 1.75 (dd, 1H, J=12.0 Hz, J=11.9 Hz); 2.05 (s, 3H); 2.7 (dd, 1H, J=12.0 Hz, J=4.6 Hz); 3.31 (dd, 0.6H, J=7.8 Hz, J=8.9 Hz); 3.5-3.75 (m, 11.4H), 3.76-4.05 (m, 8.9H); 4.43 (d, 1H, J=7.8 Hz); 4.67 (d, 0.6H, J=7.8 Hz), 5.23 (d, 0.4H, J=3.8 Hz). ¹³C NMR: 19.51, 24.78, 42.82, 54.51, 60.15, 62.83, 62.99, 65.36, 66.3, 71.1, 71.24, 72.68, 73.51, 73.78, 74.35, 74.53, 75.09, 75.24, 76.42, 76.45, 77.35, 77.39, 82.35, 82.46, 94.54, 98.37, 103.01, 105.92, 105.95, 176.21, 177.64.

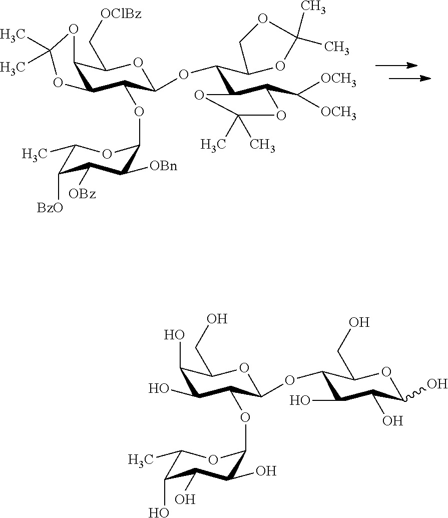

For deprotection of O-(2-O-benzyl-3,4-di-O-benzoyl-α-L-fucopyranosyl)-(1→2)-O-(6-O-(4-chlorobenzoyl)-3,4-isopropylidene-β-D-galactopyranosyl)-(1→4)-2,3:5,6-di-O-isopropylidene-D-glucose dimethyl acetal to 2′-fucosyllactose see WO 2010/115935.

1. Compounds of general formula 1 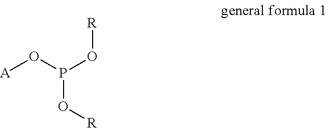
wherein A is glycosyl residue of a mono-, di- or oligosaccharide in protected form and R is selected from optionally substituted aryl or optionally substituted heteroaryl.

2. The compound according to claim 1, wherein A is a protected sialyl or fucosyl moiety, and R is optionally substituted aryl.

3. The compound according to claim 1, which is a compound of general formulae 2A or 2B 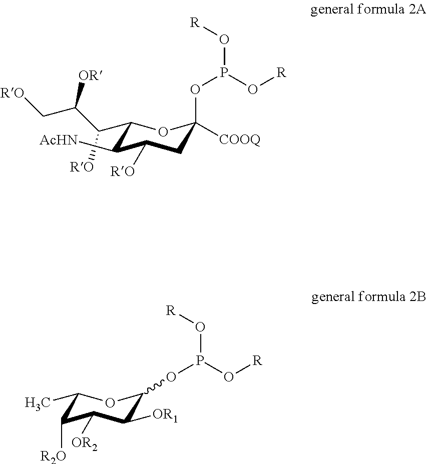
wherein R is optionally substituted phenyl, R′ is optionally substituted acyl, Q is optionally substituted alkyl, R₁ is a group removable by hydrogenolysis or optionally substituted acyl, and R₂ is a group removable by hydrogenolysis, optionally substituted acyl or two R₂ groups together form a moiety 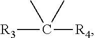
wherein R₃ and R₄, independently, are alkyl or phenyl, or wherein groups R₃ and R₄ together with the carbon atom to which they are attached form cycloalkylidene.

4. The compound according to claim 1, which is a compound of general formulae 3A or 3B 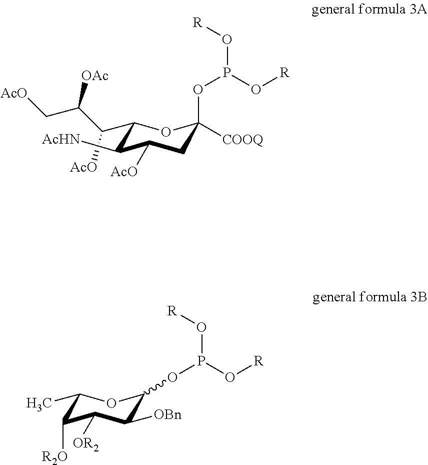
wherein Q is selected from C₁₋₆ alkyl and benzyl, preferably methyl, R is phenyl optionally substituted with alkyl, alkoxy and/or halogen, preferably methyl, methoxy and/or bromo, and R₂ is benzyl, acetyl or benzoyl optionally substituted with chloro.

5. A method for producing a compound of general formula 1 as defined in claim 1, characterized in that a compound of formula A-OH, wherein A means a protected glycosyl residue of a mono-, di- or oligosaccharide is
a) reacted with a compound (RO)₂PY wherein R is selected from optionally substituted aryl and optionally substituted heteroaryl, and Y is selected from halogen and dialkylamino, or
b) reacted with a compound PX₃ wherein X is halogen, followed by reaction with an alcohol ROH wherein R is defined as above.

6. The method according to claim 5, wherein the compound of A-OH is N-acetyl neuraminic acid tetraacetate methyl ester or 2-O-benzyl-3,4-di-O-(optionally substituted acyl)-L-fucose, PX₃ is PCl₃, and the alcohol ROH is phenol optionally substituted with alkyl, alkoxy and/or halogen, preferably methyl, methoxy and/or bromo.

7. A process for the synthesis of an oligosaccharide, characterized in that the said synthesis comprises at least the step of: coupling a compound of general formula 1 as defined in claim 1 with an acceptor of the formula B—OH, wherein B—OH means a protected mono-, di- or oligosaccharide.

8. The process according to claim 7, wherein a compound of general formulae 2A or 2B as defined in claim 3 is coupled with the acceptor B—OH to give a compound of general formulae 4A or 4B 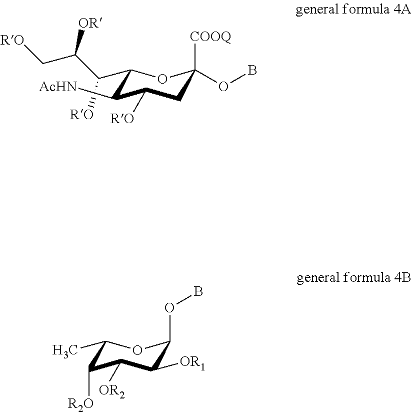
wherein R′ is optionally substituted acyl, Q is optionally substituted alkyl, R₁ is a group removable by hydrogenolysis or optionally substituted acyl, and R₂ is a group removable by hydrogenolysis, optionally substituted acyl or two R₂ groups together form a moiety 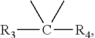
wherein R₃ and R₄, independently, are alkyl or phenyl, or wherein groups R₃ and R₄ together with the carbon atom to which they are attached form cycloalkylidene.

9. The process according to claim 7, wherein a compound of general formula 3A or 3B as defined in claim 4 is coupled with the acceptor B—OH to give a compound of general formulae 5A or 5B 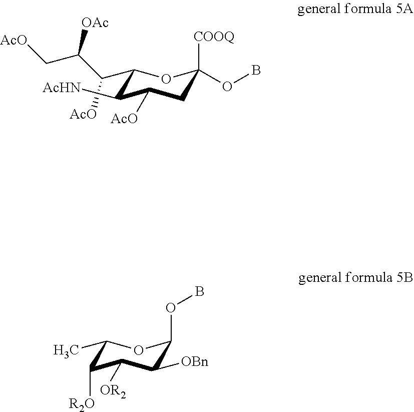
wherein Q is selected from C₁₋₆ alkyl and benzyl, preferably methyl and R₂ is benzyl, acetyl or benzoyl optionally substituted with chloro.

10. The process according to claim 7 for the synthesis of a sialylated or fucosylated human milk oligosaccharide, wherein a compound of general formula 3A or 3B as defined in claim 4 is coupled with an acceptor of the formula C—OH, wherein C—OH means a protected desialo- or defuco-human milk oligosaccharide, followed by deprotection to give the sialylated or fucosylated human milk oligosaccharide.

11. The process according to claim 10, wherein the sialylated or fucosylated human milk oligosaccharide is selected from 6′-sialyllactose, 3′-sialyllactose, 3′-sialyl-3-fucosyllactose, 2′-fucosyllactose, 3-fucosyllactose, 2′,3-difucosyllactose, sialyllacto-N-tetraoses (LST a, LST b, LST c), sialyl-fucosyllacto-N-tetraoses (FLST a, FLST b, FLST c), lacto-N-fucopentaoses (LNFP I, LNFP II, LNFP III, LNFP V), lacto-N-difucohexaoses (LNDFH I, LNDFH II, LNDFH III) and disialyllacto-N-tetraose, more preferably from 6′-sialyllactose, 3′-sialyllactose, 2′-fucosyllactose, 3-fucosyllactose, 2′,3-difucosyllactose and 3′-sialyl-3-fucosyllactose.

12. The process for the synthesis of a mixture of sialylated human milk oligosaccharides, wherein a compound of general formula 3A as defined in claim 4 is coupled with two or more protected desialo human milk oligosaccharides, followed by deprotection to give a mixture of the sialylated human milk oligosaccharides.

13. The process according to claim 12, wherein the mixture of sialylated human milk oligosaccharides comprises at least two sialylated human milk oligosaccharides selected from 6′-sialyllactose, 3′-sialyllactose, 3′-sialyl-3-fucosyllactose, sialyllacto-N-tetraoses, sialyl-fucosyllacto-N-tetraoses and disialyllacto-N-tetraose.

14. The process for the synthesis of a mixture of fucosylated human milk oligosaccharides, wherein a compound of general formula 3B as defined in claim 4 is coupled with two or more protected defuco human milk oligosaccharides, followed by deprotection to give a mixture of the fucosylated human milk oligosaccharides.

15. The process according to claim 14, wherein the mixture of fucosylated human milk oligosaccharides comprises at least two fucosylated human milk oligosaccharides selected from 2′-fucosyllactose, 3-fucosyllactose, 2′,3-difucosyllactose, 3′-sialyl-3-fucosyllactose, sialyl-fucosyllacto-N-tetraoses (FLST a, FLST b, FLST c), lacto-N-fucopentaoses (LNFP I, LNFP II, LNFP III, LNFP V), and lacto-N-difucohexaoses (LNDFH I, LNDFH II, LNDFH III).
